(19) United States
(12) Patent Application Publication
(10) Pub. No.: US 2003/0198624 A1
(43) Pub. Date: Oct. 23, 2003

(54) Method of treating allergic disease and asthma by recombinant adenovirus- and adeno-associated virus- mediated IFN-gamma gene transfer

(76) Inventors: Shyam S. Mohapatra, Tampa, FL (US); Mukesh Kumar, Norwood, MA (US); Aruna Behera, Boston, MA (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/378,985

(22) Filed: Mar. 3, 2003

Related U.S. Application Data
(60) Provisional application No. 60/360,841, filed on Mar. 2, 2002.

(51) Int. Cl. A61K 48/00; A61K 38/21

(52) U.S. Cl. 424/93.2; 424/85.5

(57) ABSTRACT

The subject invention concerns an effective therapy for asthma, including allergic disease, using cytokine gene expression therapy. The subject invention further pertains to the use of adenovirus-medicated IFN-γ(Ad-IFN-γ)gene transfer to prevent or treat allergic disease and asthma, including associated conditions such as allergen-induced airway inflammation and airway hyperresponsiveness. The subject invention includes a method for effectively attenuating allergen-induced airway inflammation and airway hyperresponsiveness by administering to the respiratory tract Ad-IFN-γ, to affect IL-12 and Stat-4 levels. The subject invention also provides compositions for gene therapy for asthma by the transfer of IFN-γ.

### CROSS-REFERENCE TO RELATED APPLICATION

This application claims the benefit of U.S. Provisional application Serial No. 60/360,841, filed Mar. 1, 2002.

### FIELD OF THE INVENTION

The subject invention pertains to the field of asthma and allergen treatment, more particularly to the use of adenovirus as a transfer vector to facilitate such treatment.

### BACKGROUND OF THE INVENTION

Allergic asthma is a chronic inflammatory disorder often characterized by airway inflammation and airway hyperreactivity (AHR). It is a leading cause of morbidity and mortality in children, adults, and the elderly. Current therapy for asthma includes treatment with bronchodilators, inhaled steroids, and leukotriene modifiers. Antigen specific immune therapy has also been used to desensitize patients to specific allergens; however, it can be ineffective for many allergic asthmatics sensitive to multiple antigens. Similarly, inhaled corticosteriods have severe adverse effects along with suppression of Th1 and Th2 cytokine responses. Also, even with currently available therapies, the incidence of asthma has continued to increase over the last two decades. Thus, an allergic asthmatic therapy is needed that induces long term effects against a broad array of antigens while providing fewer adverse effects.

Allergic asthma is caused by the disregulated production of cytokines secreted by allergen specific T-helper type 2 (Th2) cells. T helper type 1 (Th1) cells downregulate Th2 cells and Th2 pathology. As illustrated in FIG. 1, both interferon (IFN)-γ, a pleiotropic T helper type 1 (Th1) cell cytokine, and interleukin (IL)-12 induce Th1 cells. IFN-γ is capable of activating macrophages and dendritic cells to induce production of IL-12. This, however, is dependent upon the presence of IFN-γ, which provides a stimulatory signal to IL-12 (Tang, C. et al., “Th type 1-stimulating activity of lung macrophages inhibits Th2-mediated allergic airway inflammation by an IFN-gamma-dependent mechanism,” J. Immunol., 166:1471-1481 (2001)), which in turn induces Th1 responsiveness. IL-12 binds with high affinity to receptors located on T cells and natural killer (NK) cells, causing activation of tyrosine kinases and nuclear translocation of signal transducer and activator of transcription (STAT) 4, to trigger the promoter regions for the IFN-γ gene (Hasko, G., and Szabo, C., “IL-12 as a therapeutic target for pharmacological modulation in immune-mediated and inflammatory diseases: regulation of T helper 1/T helper 2 responses,” Br. J. Pharmaco., 127:1295-1304 (1999)). Studies have shown that mice deficient in STAT4 produce reduced amounts of IFN-γ in response to IL-12 and have impaired Th1 activity , indicating that STAT 4 is essential in IL-12 responses (Thierfelder, W. E. et al., “Requirement for Stat4 in interleukin-12-mediated responses of natural killer and T cells,” Nature, 382:171-174 (1996) and Kaplan, M. H., et al., “Impaired IL-12 responses and enhanced development of Th2 cells in Stat4 -deficient mice,” Nature, 382:174-177 (1996)).

Recent developments in cytokine gene therapy suggest several advantages over current therapeutic approaches used to treat allergic asthma (Barnes, P. J. et al., “Cytokine-directed therapies for asthma,” J. Allergy Clin. Immunol., 108:S72-76 (2001), Alvarez, D. et al., “Cytokine therapeutics for asthma: an appraisal of current evidence and future prospects,” Curr. Pharm. Des., 7:1059-1081 (2001), Chung, F., “Anti-inflammatory cytokines in asthma and allergy: interleukin-10, interleukin12, interferon-gamma,” Mediators Inflamm., 10:51-59 (2001), and Stirling, R. G, and Chung, K. F., “New immunological approaches and cytokine targets in asthma and allergy ,” Eur. Respir. J., 16:1158-174 (2000)). For example, the administration of recombinant IL-12 or IFN-γ have demonstrated reduced airway inflammation and airway hyperresponsiveness in murine models of allergic asthma (Schwarze, J. et al., “Local treatment with IL-12 is an effective inhibitor of airway hyperresponsiveness and lung eosinophilia after airway challenge in sensitized mice,” J. Allergy Clin. Immunol., 102:86-93 (1998), Lack, G., et al., “Nebulized but not parenteral IFN-gamma decreases IgE production and normalizes airways function in a murine model of allergen sensitization,” J. Immunol., 152:2546-2554 (1994), Gavett, S. H. et al., “Interleukin 12 inhibits antigen-induced airway hyperresponsiveness, inflammation, and Th2 cytokine expression in mice,” J Exp. Med., 182:1527-1536 (1995), and Sur, S. et al., “Mucosal IL-12 inhibits airway reactivity to methacholine and respiratory failure in murine asthma,” Exp. Lung Res., 26:477-489 (2000)). In one example, IL-12 was locally administered in the lung via systemic route (Cohen, J., “IL-12 deaths: explanation and a puzzle,” Science, 270:908 (1995) and Mohapatra, S. S., “IL-12 possibilites,” Science, 269:1499 (1995)). Unfortunately, exogenous recombinant cytokines have a short half-life in vivo, and systemic administration at high doses, especially of IL-12, may cause substantial adverse effects. In another example, plasmid DNAs (pDNA) encoding IFN-γ delivered to lung demonstrated effectiveness in modulating asthma in murine models (Li, X.M. et al., “Mucosal IFN-gamma gene transfer inhibits pulmonary allergic responses in mice,” J. Immunol., 157:3216-3219 (1996) and Dow, S. W. et al., “Systemic and local interferon gamma gene delivery to the lungs for treatment of allergen-induced airway hyperresponsiveness in mice,” Hum. Gene Ther., 10:1905-1914 (1999)). The pDNA strategy is, however, limited by the poor transfectability of lung cells, especially of lymphocytes, and hence uncertainty in terms of amount and frequency of gene delivery. Moreover, intratracheal delivery of cationic lipid and pDNA complexes is limited in the amount of plasmid that can be delivered and also requires several administrations to obtain sufficient expression of the cDNA delivered.

Adenoviruses are non-enveloped particles of size 70 nm containing a linear double stranded DNA of approximately 36,000 base pairs. They are easily prepared with high titers and can infect a wide range of cells, including non-dividing cells. An important feature of adenovirus-mediated gene expression is the ability to control the magnitude of gene expression in a dose-dependent manner.

Adeno-associated viruses have a particle diameter of 20 nm. They may integrate with relatively low efficiency, but in non-dividing cells such as muscle and neurons, they are capable of inducing high-level, long-term expression in the absence of a virus-associated inflammatory or cellular immune response.

Recently, replication-deficient adenoviruses have been used as a vehicle for transient gene expression, which permits transgene expression in a dose-dependent manner. However, treatments for asthma using adenovirus-mediated, or adeno-associated virus-mediated, IFN-γ(Ad-IFN-γ) gene transfer have not been investigated.

All documents and publications cited herein are incorporated by reference in their entirety, to the extent not inconsistent with the explicit teachings set forth herein.

### BRIEF SUMMARY OF THE INVENTION

The subject invention provides compositions and methods for the administration of cytokine gene therapy to mammals. In a preferred embodiment, the subject invention provides an adenovirus- or adeno-associated virus-mediated IFN-γ (Ad-IFN-γ) gene expression therapy for to treat and/or prevent asthma, including allergic asthma, and its associated conditions/disorders in humans.

In accordance with the subject invention, Ad-IFN-γ gene therapy modulates established inflammation and airway hyperreactivity. In one embodiment, an adenovirus or adeno-associated virus comprises a gene encoding IFN-γ, and medicaments containing it (i. e., a stabilizer), are useful for treating asthma, in particular allergic asthma. In another embodiment, Ad-IFN-γ is administered via a mucosal route, such as an intranasal, ophthalmic, or intratracheal route. In a related embodiment, Ad-IFN-γ is administered to the respiratory tract to effectively attenuate allergen-induced airway inflammation and AHR through an IL-12 and STAT-4 dependent mechanism.

In a further embodiment, intranasal delivery of Ad-IFN-γ elevates production of IFN-γ in the lung. With elevated IFN-γ production, the levels of Th2- cytokines, IL-4, IL-5, serum IgE, and eosinophilia are lowered in asthmatics exposed to allergens, such as methacholine. Using the treatment of the subject invention results in less epithelial damage, mucus plugging, and eosinophil infiltration in asthmatic lungs exposed to allergens.

The subject invention is designed to effectively attenuate established allergen-induced airway inflammation and AHR. Experimental data indicates that intranasal IFN-γ gene transfer significantly inhibits production of IL-4, IL-5, ovalbumin (OVA) specific serum IgE, airway inflammation and hyperactivity. These results demonstrate that these effects are mediated by the IL-12 and STAT-4 pathway.

### BRIEF DESCRIPTION OF THE DRAWINGS

FIG. 1 is a schematic representation of the biological process involved in inducing Th1 response.

FIGS. 2(A) through (C) illustrate the cloning and expression of recombinant Ad-IFN-γ and Ad-LacZ.

FIGS. 3(A) through (D) are graphical illustrations demonstrating the effect of Ad-IFN-γ on Th2 cytokine response in murine bronchial lymph nodes.

FIG. 4 is a graphical illustration of IL-12 mAb activity in reversing Ad-IFN-γ induced reduction in AHR and lung inflammation.

FIGS. 5(A) through (D) are photomicrographs of histologies of mice lungs treated with anti-IL-12 mAb.

FIGS. 6(A) through (D) are graphical illustrations showing the ability of Ad-IFN-γ to reverse established Th2 cytokine response in murine bronchially mph nodes.

FIG. 7 is a graphical illustration of the level of AHR in OVA-sensitized and challenged mice treated either with Ad-IFN-γ or Ad-LacZ.

FIGS. 8(A) through (C) are high-powered magnifications of bronchiole and peribronchial regions in OVA-sensitized and challenged mice.

FIGS. 8A-1, 8B-1, and 8C-1 are further enlargements of regions shown in FIGS. 8(A) through (C).

FIGS. 9(A) through (D) are graphical illustrations showing the results of anti-IL-12 mAb treatment in Ad-IFN-γ induced Th2 cytokine levels.

FIG. 10 illustrates the prevention of airway hyperresponsiveness with Ad-IFN-γ.

FIGS. 11 (A) and (B) are graphical illustrations of the effect of Ad-IFN-γ on eosinophils and antigen specific serum IgE respectively.

FIGS. 12(A) through (D) are photomicrographs of mice lung histologies.

FIGS. 13(A) through (D) are graphical illustrations showing the ability of Ad-IFN-γ to affect OVA-induced AHR and Th2 cytokine production in STAT4^{-/-} mice.

FIG. 14 is a graphical illustration demonstrating that Ad-IFN-γdoes not significantly affect OVA-induced AHR in STAT4^{-/-} mice.

FIGS. 15(A) and (B) are photomicrographs of histologies of STAT-4^{-/-} mice lungs treated with Ad-IFN-γ.

### DETAILED DISCLOSURE OF THE INVENTION

The present invention provides a method for treating asthma, in particular allergic asthma. In a preferred embodiment, replication deficient adenovirus is used for IFN-γ gene overexpression in the lung. Advantageously, Ad-IFNγ therapy provides: (1) the expression of the transgene in a dose-dependent manner at a specific tissue for an extended period of time, and (2) any overexpression is, transient and avoids undesired side effects. According to the present invention, treatment of asthma can be tailored to the needs of individuals who differ in their level of IFN-γ production and responsiveness. Further, the present invention can be used to complement therapies involving IFN-α or IFN-β.

The present invention can effectively reduce the functional and immunologic abnormalities associated with allergen sensitization and challenge. Further, the present invention can reverse allergic asthma in mammals, including humans. The term “mammals,” as defined herein, refers to any vertebrate, including human, bovine, equine, canine, feline, porcine, and ovine animals.

In a preferred embodiment, the present invention can prevent the development of allergen-induced inflammation and AHR in the respiratory tract. Further, the present invention can attenuate, or even reverse to normal, established allergen-induced airway inflammation and AHR in the respiratory tract.

In one embodiment, an adenovirus or adeno-associated virus comprises a gene encoding IFN-γ, and medicaments containing it (i.e., a stabilizer), are used in treating asthma, in particular allergic asthma. In another embodiment, Ad-IFN-γ is administered to the respiratory tract to effectively attenuate allergen-induced airway inflammation and AHR. Using the treatment of the subject invention results in less epithelial damage, mucus plugging, and eosinophil infiltration in asthmatic lungs exposed to allergens.

In a further embodiment, Ad-IFN-γ is administered intranasally. Intranasal Ad-IFN-γ transfer results in increased expression of IFN-γ and IL-12. With elevated IFN-γ production, the levels of Th2- cytokines, IL-4, IL-5, ovalbumin (OVA) specific serum IgE, and eosinophilia are lowered in asthmatics exposed to allergens, such as methacholine.

The subject invention is designed to effectively attenuate established allergen-induced airway inflammation and AHR. Experimental data indicates that intranasal IFN-γ gene transfer significantly inhibits production of IL-4 , IL-5, ovalbumin (OVA) specific serum IgE, airway inflammation and hyperactivity. These results demonstrate that these effects are mediated by the IL-12 and STAT-4 pathway.

Following is an example illustrating procedures for making and practicing the invention. This example should be construed to include obvious variations and is not limiting. Unless noted otherwise, all solvent mixture proportions are by volume and all percentages are by weight.

The methods traditionally used in molecular biology, such as preparative extractions of plasmid DNA, centrifugation of plasmid DNA in a cesium chloride gradient, agarose or acrylamide gel electrophoresis, purification of DNA fragments by electroelution, phenol or phenol/chloroform extraction of proteins, ethanol or isopropanol precipitation of DNA in a saline medium, transformation in Escherichia coli, and the like, are well known to a person skilled in the art and are amply described in the literature (Maniatis T. et al., “Molecular Cloning, a Laboratory Manual,” Cold Spring Harbor Laboratory, Cold Spring Harbor, N.Y., (1982); Ausubel, F. M. et al. (eds), “Current Protocols in Molecular Biology,” John Wiley & Sons, New York, (1987)).

### EXAMPLE 1

In this Example, the effectiveness of Ad-IFN-γ treatment for allergic asthma was assessed.

Materials

In this Example, a BALB/c mouse model of established allergic asthma was used to examine the impact of intranasal IFN-γ gene transfer on allergic inflammation immunopathology and airway hyperactivity. Female 6-8 weeks old wild type and STAT4^{-/-} BALB/c mice from Jackson Laboratory (Bar Harbor, Me.) were maintained in pathogen-free conditions.

Cloning and Recombination of Adenoviral Vecotrs

Murine IFN-γ cDNA was cloned into adenoviral transfer vector pSHUTTLE-CMV (Stratagene, Calif.) at Kpril and Xhol sites. The left and right arms of pSHUTTLE-CMV vector contains Ad5 nucleotides 35,931-35,935 and 3,534-5,790, which mediate homologous recombination with pAdEasy-1 vector in E.coli, plus inverted terminal repeat (TTR) and packaging signal sequences (nucleotides 1-480 of Ad5) required for viral production in mammalian cells. PAdEasy-1 adenoviral plasmid (Stratagene, Calif.) contains all Ad5 sequences except nucleotides 1-3,533 (encompassing the E1 gene) and nucleotides 28,130-30,820 (encompassing E3).

For generation of recombinant adenovirus plasmid, pSHUTTLE-CMV-IFN-γ/LacZ plasmids were linearized with PmeI and co-transformed with pAdEasy-1 plasmid into recombination proficient BJ5183 cells. The recombination was confirmed by PacI digestion. The recombined clones were re-transformed in DH5α cells for large-scale plasmid purification.

Generation and Purification of Recombinant Adenovirus

HEK293 cells, which produce the deleted E1 genes in trans, were transfected with 4 μg of PacI digested recombinant adenovirus plasmid DNA with LIPOFECTIN (Life Technologies, MD). Cells were harvested 7-10 days post-transfection, resuspended in PBS and recombinant virus was collected by 3-4 freeze-thaw cycles. The recombinant virus expressing murine INF-γ and LacZ were termed Ad-IFN-γ and Ad-LacZ, respectively. The viruses were amplified by infecting fresh HEK-293 cells. Viruses were further purified by CsCl banding, a process that is well known to those skilled in the art and is disclosed by Becker et al., “Use of recombinant adenovirus for metabolic engineering of mammalian cells,” Methods Cell Biol., 43:161-189 (199). The viral band was extracted and CsCl removed by passing through CENTRICON-100 columns (Millipore, Mass.). Viral plaque assay was performed following known protocols, such as those described by He et al., “Simplified system for generating recombinant adenoviruses,” Proc. Nat'l. Acad. Sci. U.S.A., 95:2509-2514 (1998).

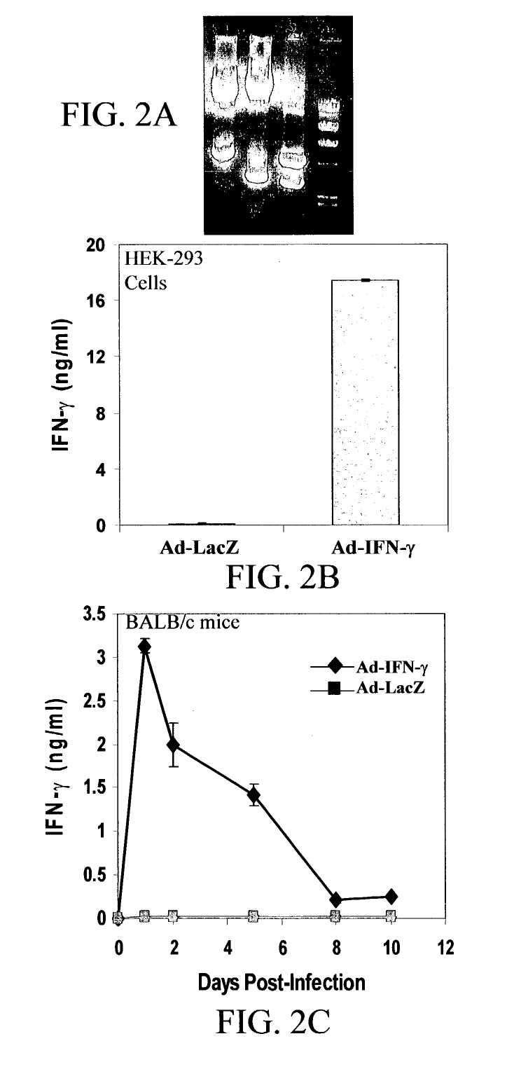

Referring now to FIGS. 2(A) through (C), cloning and expression of recombinant Ad-IFN-γ and Ad-LacZ are shown. Complete cDNA of murine IFN-γ and E.coli LacZ were cloned into the adenoviral shuttle vector and the recombinant adenoviruses were isolated from the rec+ E. coli and verified by restriction digestion, as illustrated in FIG. 2A. The expression of the purified recombinant adenoviruses were monitored in HEK-293 cells 24 h after infection. The supernatants from the infected cells were analyzed for IFN-γ expression by ELISA, as illustrated in FIG. 2B. The results in FIG. 2B demonstrate that HEK-293 cells infected with AD-IFN-γ, and not Ad-LacZ, produce 17.4 ng/ml of IFN-γ after 48 h of transfection. Time course expression of IFN-γ in vivo was examined in BALB/c mice. IFN-γ expression was measured by ELISA in broncho-alveolar lavage (BAL) fluid collected on days 1, 2, 5, 8 and 10 from mice (n=4) administered with Ad-IFN-γ or Ad-LacZ (control). IFN-γ expression peaked on day 1 in BAL fluid from Ad-IFN-γ administered mice, as illustrated in FIG. 2C, and then slowly declined over the next few days and reached basal level by day ten. In contrast, no IFN-γ was detected in the BAL from mice receiving the control virus, Ad-LacZ, or HEK-293 cells infected with Ad-LacZ. These results show that the infection by adenovirus itself does not induce expression of IFN-γ.

Treatment According to the Subject Invention: Prevention of AHR

BALB/c wild type or STAT4^{-/-} mice were sensitized i.p. with ovalbumin (OVA) (50 μg) absorbed by 2 mg of aluminum potassium sulfate (alum) on days 1 and 15. On day 29, 1.0×10⁸ PFU of Ad-IFN-γ or Ad-LacZ was administered i.n., followed by intranasal challenge with 50 μg of OVA on days 30, 31, and 32. For depletion of IL-12, group of BALB/c mice were intranasally administered with 200 μg of anti-IL-12 mAb or isotype matched control mAb in the mornings of days 30, 31, and 32 and challenged on the same days in the evenings with 50 μg of OVA. The day following last challenge, day 33, AHR was measured in conscious mice to increasing concentrations of methacholine. On day 34, mice were bled, and then sacrificed, bronchial lymph nodes and lungs were removed and single cell suspension of bronchial lymph node cells were prepared and cultured in vitro either in the presence of 100 μg/ml OVA or medium alone.

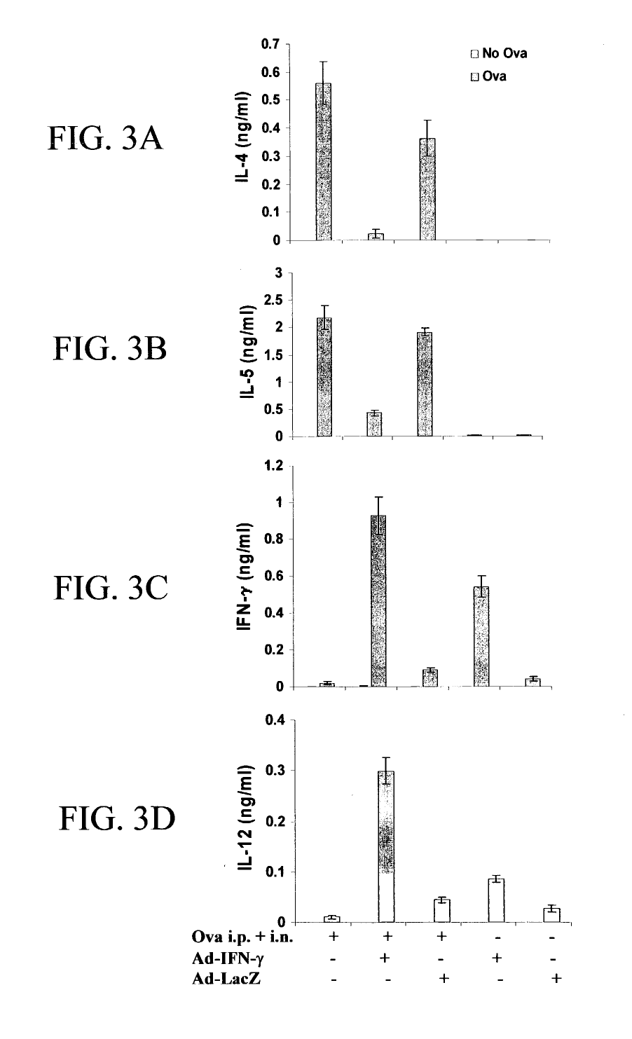

The results of the immunization and treatment strategy is schematically shown in FIGS. 3(A) through (D). To examine the effect of intranasal Ad-IFN-γ administration on the cytokine production in the lung, single cell suspensions of bronchial lymph nodes were cultured in medium alone or with 100 μg/ml OVA for 48 h and the amounts of IL-4 , IL-5, IFN-γ and IL-12 in the supernatant were quantified. Ad-IFN-γ treatment significantly decreased OVA-induced production of IL-4 (p<0.01) and IL-5 (p<0.01) and increased OVA-induced IFN-γ (p<0.01) and IL-12 (p<0.01) in the culture supernatants, when compared to control group that did not receive Ad-IFN-γ or the group receiving Ad-LacZ. Groups of control mice, which were not OVA-sensitized but administered with Ad-IFN-γ and challenged showed significant expression of IFN-γ (p<0.01) and IL-12 (p<0.01), but neither IL-4 nor IL-5, compared to those treated with Ad-lacZ. None of these cytokines could be detected in the culture supernatants in absence of OVA stimulation. These results suggest that Ad-IFN-γ treatment promotes a Th1-like response and suppresses a Th2-like response.

Treatment According to the Subject Invention: Reversal of Established AHR

Mice sensitized i.p. with 50 μg OVA on days 1 and 15, were intranasally challenged with 50 μg of OVA on days 29 and 30. On day 44, 1.0×10⁸ PFU ofAd-IFN-γ or Ad-LacZ was administered i.n., followed by booster challenge i.n. with 50 μg of OVA i.n. on days 45, 46, and 47 and AHR was measured on day 48. Mice were bled and sacrificed on day 49.

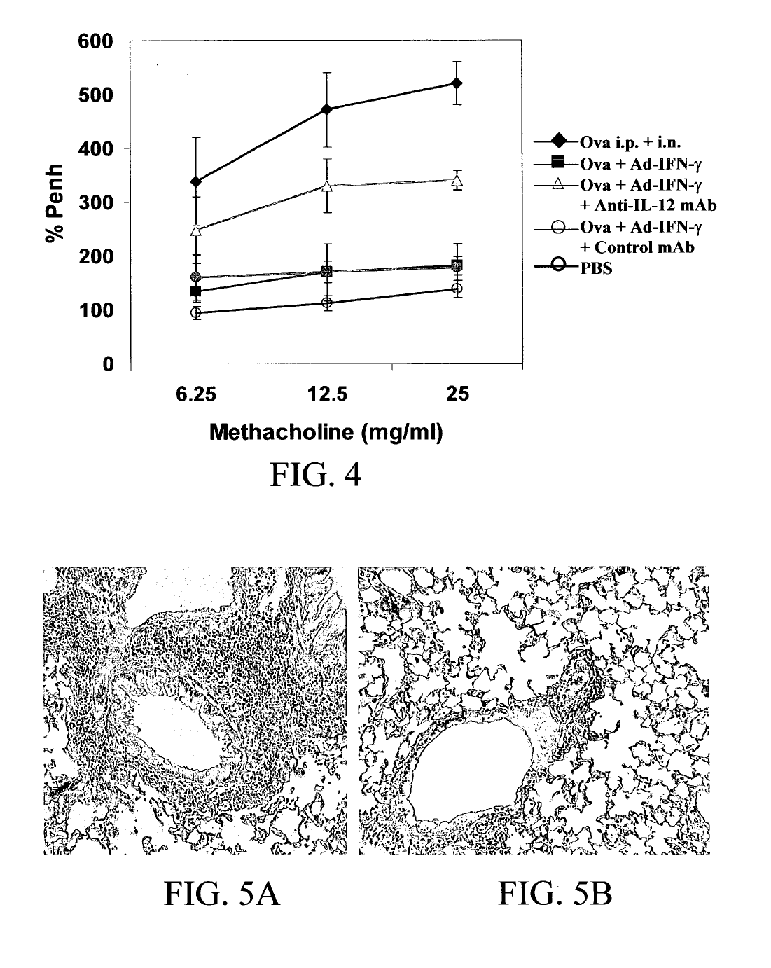

As illustrated in FIG. 4, OVA-sensitized and challenged mice that received Ad-IFN-γ and isotype matched control mAb showed significantly lower AHR (p<0.01) when compared to the OVA-sensitized and challenged group. Intranasal administration of anti-IL-12 mAb significantly reversed the reduction in AHR conferred by Ad-IFN-γ. Mice receiving Ad-IFN-γ and anti-IL-12 mAb showed significantly higher AHR (p<0.05) when compared to mice receiving Ad-IFN-γ alone or Ad-IFN-γ +control mAb. There was no significant difference between the OVA sensitized and challenged group and the group receiving Ad-IFN-γ and anti-IL-12 mAb.

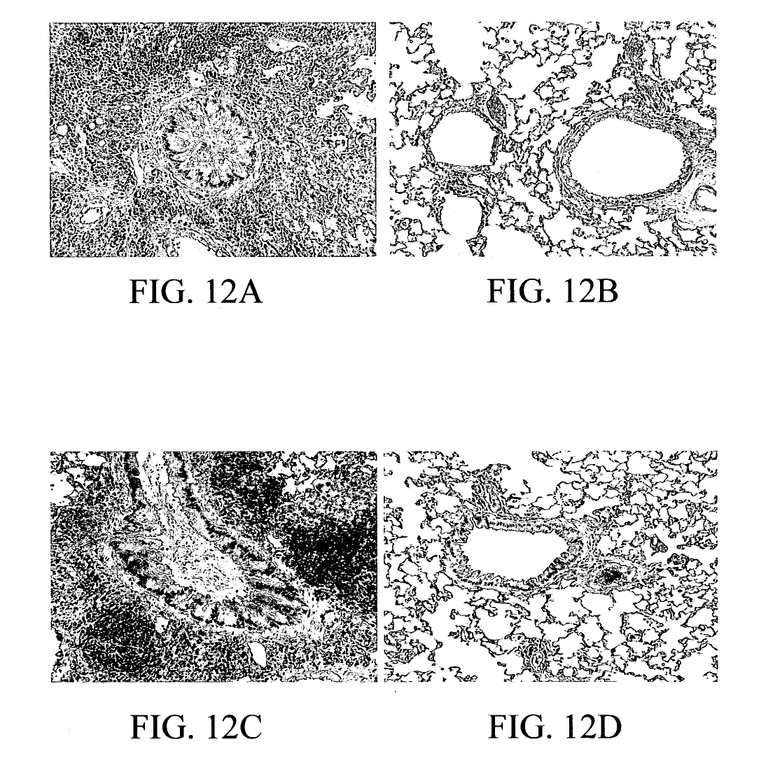

To examine the effect of anti-IL-12 mAb on Ad-IFN-γ-induced lung histology in OVA-sensitized mice, lung sections from Ad-IFN-γ-treated mice administered with anti-IL-12 mAb or an isotype matched control antibody were compared. As illustrated in FIGS. 5(A) and (B), Ad-IFN-γ mediated protection from lung inflammation was also reversed in the group receiving anti-IL-12 mAb with the presence of increased infiltration of mononuclear cells and polymorphs in the interstitial and peri-broncho-vascular regions, whereas the group receiving the Ad-IFN-γ+isotype matched control mAb showed significantly reduced lung pathology (FIG. 5(B)) similar to Ad-IFN-γ treated group (FIG. 12(B)). Together these results show that the Ad-IFN-γ-induced reduction in AHR and lung pathology is predominantly dependent on IL-12 expression.

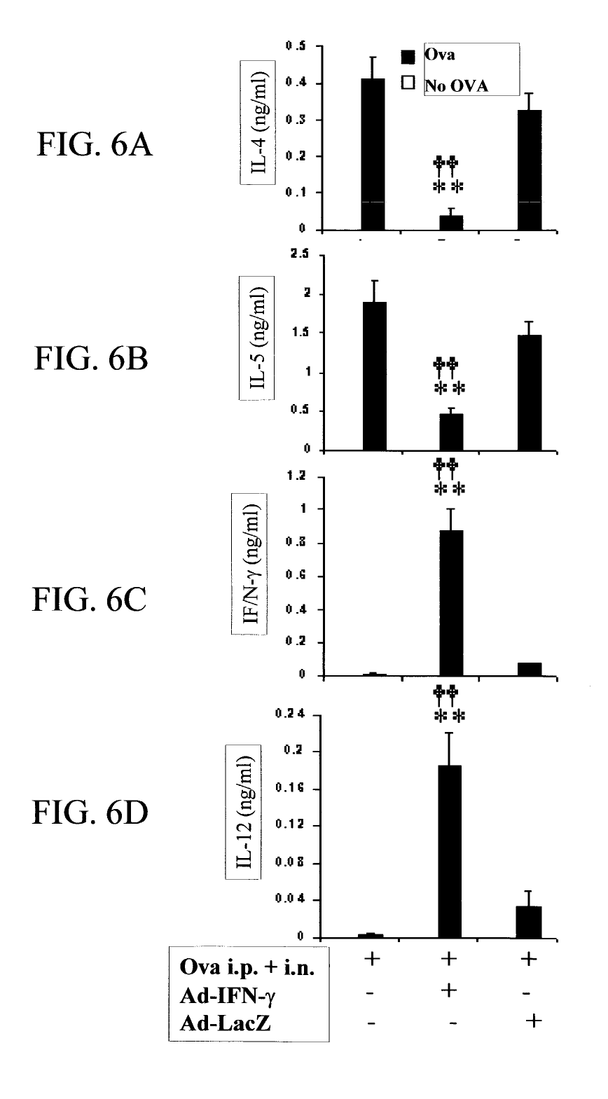

To determine whether Ad-IFN-γ can reverse the established Th2 response, mice were sensitized with OVA twice at an interval of 15 days and then challenged i.n. on days 29 and 30 and again on days 45-47. As illustrated in FIGS. 6(A) through (D), intranasal administration of Ad-IFN-γ to OVA sensitized and challenged mice significantly decreased OVA-induced production of IL-4 (p<0.01) and IL-5 (p<0.01) and increased OVA-induced IFN-γ (p<0.01) and IL-12 (p<0.01) in the bronchial lymph node cells, when compared to those from OVA-sensitized and challenged mice and the group receiving Ad-LacZ. These results show that Ad-IFN-γ treatment can reverse an established Th2 response.

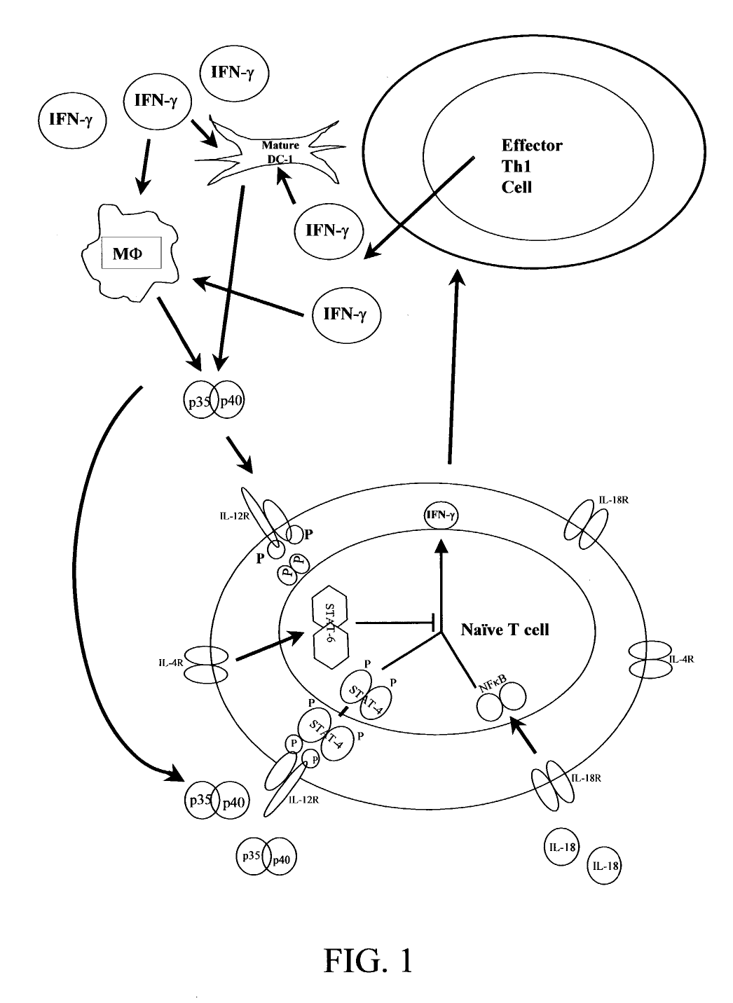

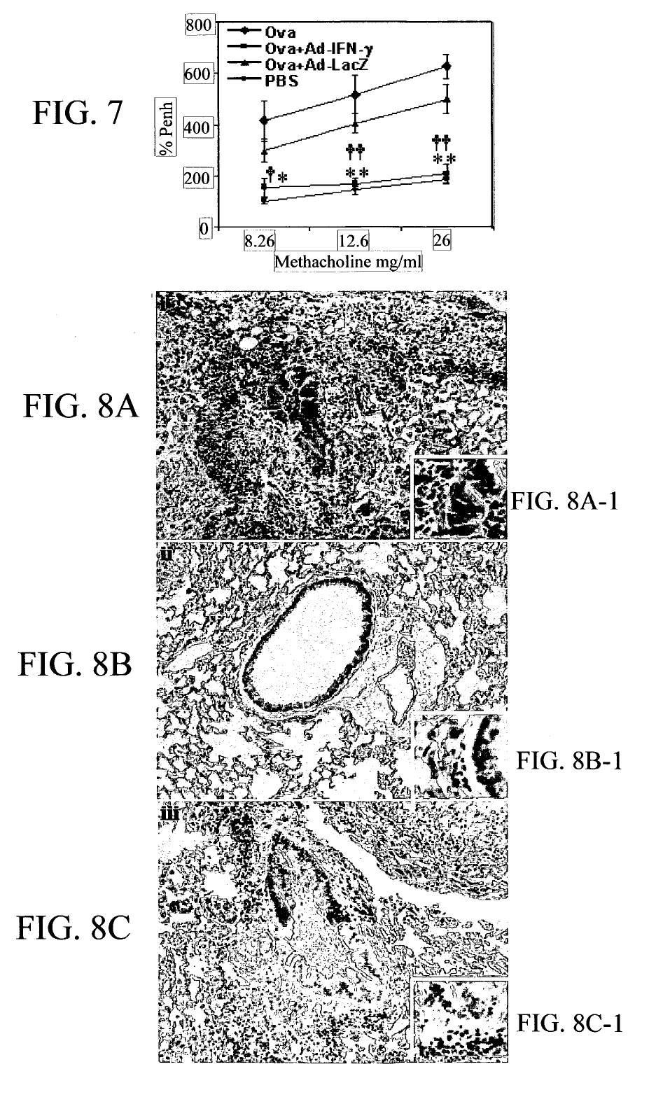

To examine whether Ad-IFN-γ administration can reverse established AHR, mice were treated with Ad-IFN-γ after i.n. OVA challenge. As illustrated in FIG. 7, mice receiving Ad-IFN-γ following OVA-sensitization and challenge showed significantly (P<0.01) lower AHR when compared to mice that did not receive Ad-IFN-γ or received Ad-LacZ. This demonstrates that Ad-IFN-γ administration not only inhibits development of AHR but also reverses established AHR in murine models. Treatment with Ad-IFN-γ to OVA-sensitized and challenged mice significantly reduced epithelial damage, infiltration of mononuclear cells and polmorphs in the interstitial and peri-broncho-vascular regions, as illustrated in FIG. 8(B) and in the enlarged region in FIG. 8B-1, when compared to OVA-sensitized and challenged mice, as illustrated in FIG. 8(A) and in the enlarged region in FIG. 8A-1, or mice treated with Ad-LacZ, as illustrated in FIG. 8(C) and in the enlarged region in FIG. 8C- 1. Taken together, these results show that administration of Ad-IFN-γ significantly reverses AHR and lung inflammation associated with established allergic condition in OVA sensitized and challenged mice.

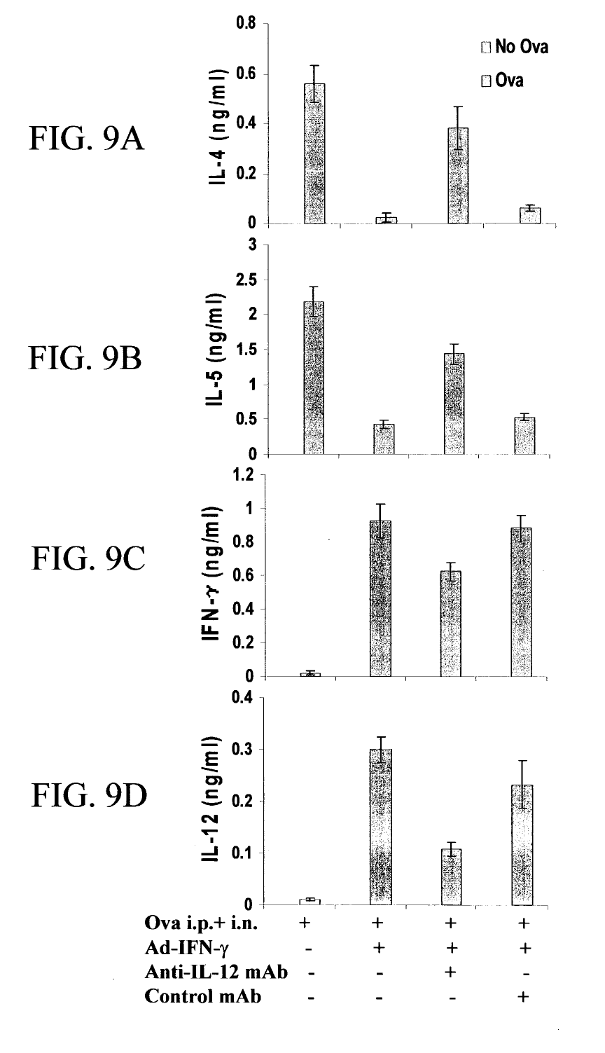

To investigate the mechanism of IFN-γ action in reducing allergic inflammation and AHR, mice (n=8) were administered i.n. anti-IL-12 mAb or isotype matched control mAb (n=8) for three consecutive days. As illustrated in FIGS. 9(A) through (D), the effect of the treatment of anti-IL-12 mAb on the T helper cell response was examined by analyzing the cytokine expression pattern in the bronchial lymph node cells cultured either in medium alone or induced with OVA. The group that received anti-IL-12 mAb produced, significantly higher levels of IL-4 and IL-5 compared to the group that received Ad-IFN-γ and the control mAb (p<0.01) and the group that received Ad-IFN-γ alone (p<0.01). The differences in the expression of IL-4 and IL-5 were not significant between OVA-sensitized and challenged mice and those receiving Ad-IFN-γ+anti-IL-12 mAb. These results show that blocking IL-12 by anti-IL-12 mAb significantly blocks Ad-IFN-γ effect.

Treatment According to the Subject Invention: Measurement of Airway Hyperresponsiveness

Airway hyperresponsiveness to inhaled methacholine was measured using the whole body PLETHYSMOGRAPH (Buxco, Troy , N.Y.), using known procedures such as those disclosed by Matsuse et al., “Recurrent respiratory syncytial virus infections in allergen-sensitzed mice lead to persistent airway inflammation and hyperresponsiveness,” J. Immunol., 164:6483-6492 (2000).

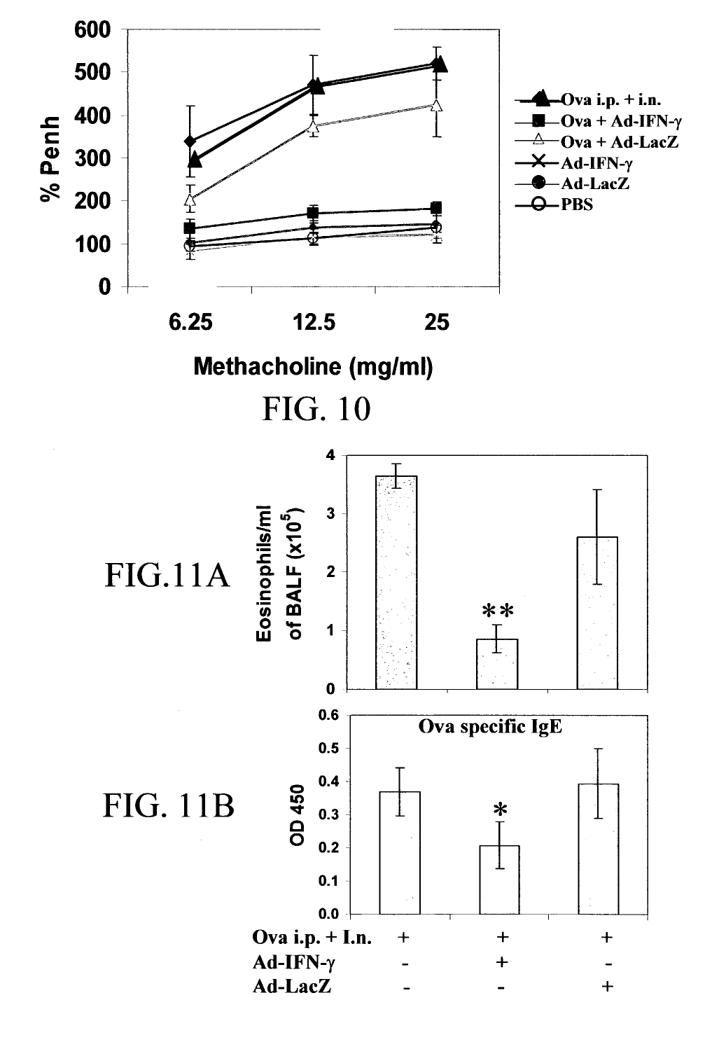

To examine the effect of Ad-IFN-γ administration on allergic inflammation and asthma, the AHR, lung eosinophilia and IgE was measured in mice. Groups (n=6) of mice immunized i.p. twice with 50 μg/ml of OVA, were treated with either Ad-LacZ (n=6) or Ad-IFN-γ (n=8) and then challenged i.n. with OVA on days 30, 31 and 32. Their AHR was measured by whole-body plethysmography and compared with PBS as baseline. Both groups of control mice, those which received Ad-LacZ virus i.n. on day 29 prior to OVA challenge, and those which were OVA sensitized and challenged without any treatment, exhibited significant airway hyperresponsiveness to increasing concentrations of methacholine, as illustrated in FIG. 10. No significant difference was observed between Ad-LacZ treated and untreated mice. In marked contrast, administration of Ad-IFN-γ i.n. prior to OVA challenge significantly reduced (p<0.01) the development of AHR compared to the control groups. There was no significant difference between Ad-IFN-γ treated mice and those treated with Ad-LacZ or Ad-IFN-γ without OVA sensitization. This effect was IFN-γ-specific, as the mice receiving Ad-LacZ did not significantly reduce AHR.

As illustrated in FIGS. 11(A) and (B), treatment with Ad-IFN-γ reduced the number of eosinophils in the BALF from 3.65×10³ to 0.863×10³ (<0.01) when compared to the OVA-sensitized and challenged group or the group receiving Ad-LacZ, indicating that the reduction in eosinophils is specific to IFN-γ expression. Ad-IFN-γ administration also significantly reduced (p<0.05) OVA-specific serum IgE levels when compared to the OVA-sensitized and challenged group and the group receiving Ad-LacZ. These results show that Ad-IFN-γ treatment significantly reduces AHR, lung eosinophilia and allergen specific IgE.

As illustrated in FIGS. 12(A) through (D), lung inflammation was examined from formalin fixed, hematoxylin and eosin (H & E) stained lung sections from all treatment groups. The Ad-IFN-γ treated group exhibited reduced epithelial damage, less infiltration of mononuclear cells and polymorphs in the interstitial and peri-broncho-vascular regions (FIG. 12(B)) compared to the control group, which either received Ad-LacZ (FIG. 12(C)) or the OVA-sensitized and -challenged group (FIG. 12(A)). Mice treated with Ad-IFN-γ did not show large inflammatory regions in the (FIGS. 12(B) and (D)) mice, however, very few inflammatory cells appear around the peri-broncho vascular region in Ad-IFN-γ treated mice as seen in FIG. 12(B). The group receiving Ad-IFN-γ and i.n. OVA alone (not OVA i.p.) had nearly normal lung morphology but with thickened epithelial layer (FIG. 6D). A similar result was obtained with the group receiving Ad-LacZ and OVA i.n. (but not OVA i.p.). Thus, the recombinant adenovirus itself did not induce lung pathology.

Treatment According to the Subject Invention: Bronchial lymph Node Culture and Assay for cytokines

Single cell suspensions of bronchial lymph nodes (3×10⁶ cells/well of 24 well plate) were restimulated in vitro in the presence or absence of 100 μg/ml OVA. Supernatants were collected after 48 h for cytokine ELISAs. ELISA for IL-4, IL-5, IL-12 and IFN-γ were performed using kits from R & D SYSTEMS (Minneapolis, Minn.) following manufacturer's protocol.

Treatment According to the Subject Invention: OVA-specific IgE Analysis

To determine the OVA-specific IgE, microtitre plate was coated overnight at 4° C. with 100 μl of OVA (5 μg/ml). Non-specific sites were blocked following three washes with PBST (0.5% Tween-20 in PBS). Mouse sera were incubated to the antigen-coated wells and bound IgE was detected with biotinylated anti-mouse IgE (02112D; Pharmingen, Calif.). Biotin anti-mouse IgE (021222D) reacts specifically with the mouse IgE of Igh^{a} and Igh^{b} haplotypes and does not react with other IgG isotypes. Diluted streptavidin-peroxidase conjugate was added, the bound enzyme detected with TMB, and the absorbance read at 450 nm.

Treatment According to the Subject Invention: Lung Histology

Mice were sacrificed within 24 hours after the last challenge, and lung sections were subjected to paraffin embedding. Lung inflammation was assessed after the sections were stained with hematoxylin and eosin. The results of the study described in Example 1 show the following:

1. Intranasal delivery of recombinant adenovirus expressing IFN-γ expresses high level IFN-γ in murine lung without causing any significant inflammatory response;

2. Ad-IFN-γ significantly reduced OVA-induced airway hyperresponsiveness;

3. Ad-IFN-γ promotes Th1 cytokine production and reduces Th2 cytokine production in thoracic lymph nodes;

4. Ad-IFN-γ decreases the levels antigen-specific serum IgE antibodies and number of eosinophils bronchoalveolar lavage fluid;

5. Ad-IFN-γ restores normal lung histology in OVA -sensitized and -challenged mice;

6. IFN-γ mediates its protective anti-allergic response via the expression of IL-12, as concomitant administration of anti-IL-12 mAb significantly reverses the protective response of IFNγ, and

7. IFN-γ mediated anti-allergic response is dependent on the expression of STAT-4.

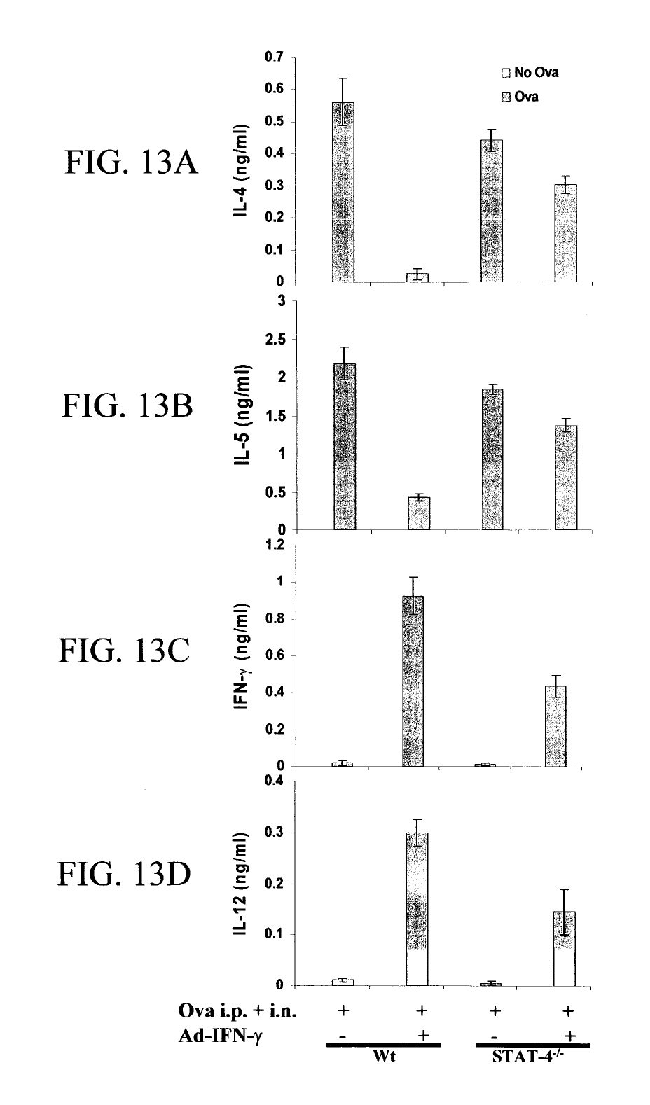

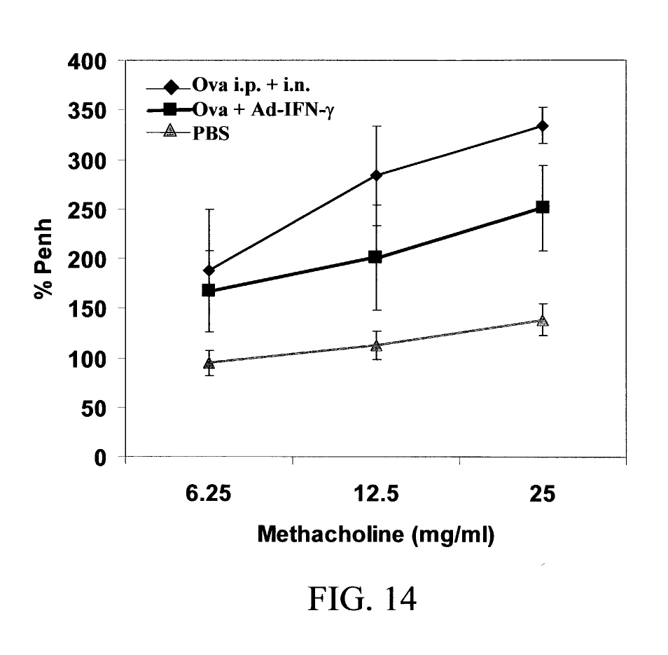

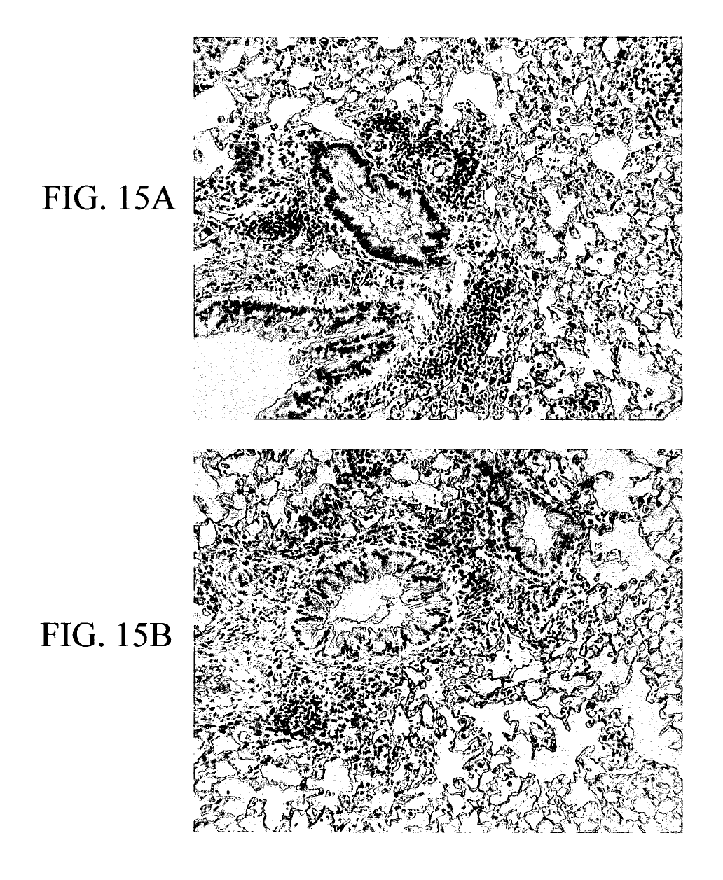

To further confirm the requirement of IL-12 in mediating the anti-inflammatory response of Ad-IFN-γ, the study was extended to STAT-4^{+/+} and STAT-4^{-/-} mice. Mice were treated with Ad-IFN-γ (n=8). Examination of the cytokine profiles from the bronchial lymph node cultures of STAT4^{-/-} mice upon stimulation in vitro with OVA showed a significantly elevated expression of IL-12 (p<0.05) and IFN-γ (p<0.01), but no significant reduction in Th2 cytokines (IL-4 and IL-5) in mice receiving Ad-IFN-γ, when compared to OVA-sensitized and challenged mice, as illustrated in FIGS. 13(A) through (D). There was no significant difference in AHR between the group administered with Ad-IFN-γ and OVA-sensitized and challenged STAT4^{-/-} group, as illustrated in FIG. 14, even though a moderate decrease in AHR was observed in mice receiving Ad-IFN-γ. An analysis of lung sections revealed that both the OVA sensitized and challenged group and the group receiving Ad-IFN-γ showed similar lung pathology with increased infiltration of mononuclear cells in the peri-broncho-vascular region (FIGS. 15(A) and (B)). Similarly no significant difference was observed in OVA specific IgE levels between the two groups. These data indicate that the reduction in AHR, Th2 cytokines, and lung inflammation conferred by Ad-IFN-γ is predominantly STAT-4 dependent.

The present invention provides a method for enhancing the expression of IFN-γ in humans. As is known to the skilled artisan, the T helper cell differentiation pathway functions similarly in both mice and humans. Current data regarding certain gene therapies in immune responses suggest that results shown in murine models can analogously and successfully be induced in humans (Payne L G, Fuller D H, Haynes J R., “Particle-mediated DNA vaccination of mice, monkeys and men: looking beyond the dogma,” Curr Opin Mol Ther, (5):459-66 (2002)). Moreover, IFN-γ has demonstrated an ability to decrease (i) IL-13− induced goblet cell hyperplasia and eosinophilia by diminished IL-13 signaling through upregulation of the IL-13Rα2 decoy receptor (Ford et al., 2001; Daines et. al., 2002); (ii) LTC4 and human macrophages (Boraschi et al., 1987; Thivierge et al., 2001), in human peripheral blood lymphocytes after wasp venom immunotherapy (Pierkes et al., 1999), and in leukocytes of pollinosis patients (Krasnowska et al., 2000); and (iii) TGF-β, and procollagen-I and -III, which cause fibrosis and airway remodeling (Gurujeyalakshmi et al., 1995; Minshall et al., 1997). This invention can be used to enhance IFN-γ expression both prophylactically and/or therapeutically to threat allergic diseases, including allergic asthma.

Inasmuch as the preceding disclosure presents the best mode devised by the inventor for practicing the invention and is intended to enable one skilled in the pertinent art to carry it out, it is apparent that methods incorporating modifications and variations will be obvious to those skilled in the art. As such, it should not be limited thereby but should include such aforementioned obvious variations.

We claim:

1. A method of enhancing IFN-γ expression to regulate the production of cytokines secreted by Th2 cells comprising administering an adenovirus comprising an IFN-γ gene in a mammal.

2. The method according to claim 1, wherein the adenovirus comprising the IFN-γ gene is administered to humans.

3. The method according to claim 1, wherein the adenovirus is administered to cells of the respiratory tract.

4. The method according to claim 1, wherein the adenovirus is in a medicament.

5. A method if enhancing IFN-γ expression to regulate the production of cytokines secreted by Th2 cells comprising administering an adeno-associated virus comprising an IFN-γ gene in a mammal.

6. The method according to claim 5, wherein the adenovirus comprising the IFN-γ gene is administered to humans.

7. The method according to claim 5, wherein the adenovirus is administered to cells of the respiratory tract.

8. The method according to claim 5, wherein the adenovirus is in a medicament.

9. A pharmaceutical composition comprising an adenovirus comprising an IFN-γ gene.

10. A pharmaceutical composition comprising an adeno-associated virus comprising an IFN-γ gene.
